(19) United States
(12) Patent Application Publication
(10) Pub. No.: US 2005/0054932 A1
(43) Pub. Date: Mar. 10, 2005

(54) Ultrasound transducer

(75) Inventors: Michael Kochan, Ottendorf-Okrilla (DE); Gerry Schroeter, Dresden (DE)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Sick Engineering GmbH, Ottendorf-Okrilla (DE)

(21) Appl. No.: 10/936,979

(22) Filed: Sep. 8, 2004

(30) Foreign Application Priority Data
Sep. 9, 2003 (DE) 103 41 422.3

(51) Int. Cl. A61B 8/00

(52) U.S. Cl. 600/459

(57) ABSTRACT

An ultrasound transducer has a housing, at least one piezoelectric element, and a membrane emitting or receiving ultrasound. A mass ring is arranged at an edge portion of the membrane for the dampening of undesirable resonances. To provide an ultrasound transducer with the smallest possible dimensions, the ultrasound transducer is configured as a lengthwise or longitudinal oscillator, the mass ring is separate from the membrane and is joined to both the edge portion of the membrane and the inside of the housing. A ring-shaped dampening element is attached to the inside of the housing and is located next to the mass ring.

### BACKGROUND OF THE INVENTION

The invention concerns an ultrasound transducer with a piezoelectric element, a membrane for emitting and receiving ultrasound, and a mass ring coupled to the membrane for dampening undesirable resonances.

DE 100 40 344 A1 discloses an ultrasound transducer that is used to generate and detect ultrasonic signals and enables a reciprocal conversion of electrical oscillations into acoustic oscillations. Such ultrasound transducers are used, for example, in gas flow meters. A pair of ultrasound transducers are arranged to define a metering path which is non-perpendicular to the longitudinal axis. The metering principle of such transducers determines a transit time difference between two ultrasonic signals, one of which has a component in the flow direction and the other one a component opposite the flow direction. From the measured time difference, one can calculate the flow velocity, while also considering the influence of the geometry.

FIG. 2 of this application shows a flow meter with ultrasound transducers 16 and 18 that generate and measure the ultrasonic signals and are inserted into a pipe 12 or the pipe wall by means of adapter flanges 24 and 26, which form so-called transducer pockets extending into pipe 12 or its wall. The adapter flanges are either welded on or they form an integral part of the meter housing, if the housing is cast. Since the ultrasound transducers 16 and 18 are installed at a certain angle (usually 45°), a cavity 28 will always form, which constitutes a flow disturbance. This disturbance exists regardless of how deeply the ultrasound transducer is inserted, and whether centered, retracted or projecting. The disturbance increases with the diameter of the sensor and, associated therewith, the size of the transducer pocket in relation to the diameter of the metering cell. The eddies that are formed as a result thereof cannot be entirely analytically calculated and will depend on any preexisting flow disturbances upstream from the flow meter as well as the velocity of the flow (Reynold's number). Resulting errors are generally determined by calibration and are taken account of by employing a usually nonlinear correction function. Since a given calibration only covers a particular range of Reynold's numbers and a specific installation configuration, a residual error is created when operating conditions are changed, which is usually the case.

Moreover, in practice, ultrasound transducers are arranged in multiple-path layouts to detect flow asymmetries. The realizable number of paths is dictated by the available installation space and is limited by the size of the transducer. To enhance the accuracy of flow metering, it is therefore advantageous to keep the dimensions of the transducer as small as possible.

There is further the danger that a variety of deposits can accumulate in the resulting cavity, which can undesirably influence the measurement accuracy. This accumulation of deposits increases as the cavity becomes bigger.

Although it is desirable to make the transducers as small as possible, there are functional (e.g. transmission technology) and technological limits (e.g. the feasibility and efficiency of miniature fabrication) which stand in the way of further size reductions. Ultrasound transducers for use in gases are preferably relatively large in relation to the size of the gas meter due to the relatively low operating frequencies.

Due to their dimensions and configurations, prior art transducers limit the attainable measurement accuracy because of excessive flow disturbances and/or because they do not allow a multiple-path layout of sensors due to space limitations. The size of the sensors significantly affects the overall design of a complete meter and creates additional problems, e.g. inadequate compressive strength and high material requirements and weight, which adversely impacts production costs. The handling of the meters during fabrication, transportation, installation, maintenance and repair also becomes more cumbersome.

U.S. Pat. No. 4,162,111 discloses an ultrasound transducer with a piezoelectric element, pressed by a spring against a membrane emitting the ultrasound. The membrane has an enlarged, ring-like edge along its margin that is formed as a single piece with the rest of the membrane, has a larger mass, and due to its mass serves to dampen disturbing frequencies. In its longitudinal direction, the edge is kept as short as possible in its lengthwise extent, and it is arranged at the level of the piezoelectric element.

### SUMMARY OF THE INVENTION

It is therefore an object of the present invention to provide an improved ultrasound transducer which significantly lessens the earlier mentioned drawbacks encountered with prior art transducers, in that the transducer is fabricated with the smallest possible dimensions.

The ultrasound transducer of the invention has a housing, at least one piezoelectric element and a membrane emitting or picking up the ultrasound. A mass ring is arranged along the margin of the membrane. Furthermore, the invention contemplates to configure the ultrasound transducer as a longitudinal oscillator and to separate the mass ring from the membrane. The mass ring is separate from and joined to the membrane and the housing, and on its inside the housing has a dampening element that is configured as a ring and situated adjacent to the mass ring.

In this embodiment of the invention, the diameter of the transducer can be kept very small relative to the working frequency. That is, for the same working frequency, the transducer is smaller than previously known transducers, while sufficient stiffening and dampening of the oscillating system can be employed to avoid secondary resonances. This is an important effect due to having the mass ring separated from the membrane, as the inventor has discovered. This effect can be enhanced by arranging a dampening element next to the mass ring, which further reduces parasitic secondary resonances.

It is advantageous for the mass ring to be screwed to the membrane, so that the mass ring and the membrane are firmly joined together in definite relative positions to each other. The same applies to joining of the mass ring to the housing.

Ultrasound transducers are often used in flow meters operating in corrosive and/or dangerous media, as well as under high pressures and temperatures. The membrane and the housing are advantageously welded together at the level of the mass ring. This ensures an absolute tightness of the transducer.

To assure that the dampening element securely engages the housing, which parasitically conducts ultrasound, and its dampening effect can be optimally used, the dampening element is made of an elastic material, preferably a rubber-like material.

In one embodiment of the invention, the separate mass ring can be made of a single piece with the housing. The term “separate” is merely intended to mean that the mass ring is provided separately of the membrane, which is an important feature of the invention for achieving advantageous dampening.

### BRIEF DESCRIPTION OF THE DRAWINGS

FIG. 1 is a cross-section through an ultrasound transducer constructed in accordance with the invention;

FIG. 2 shows a metering layout for metering a flow of a fluid which makes use of the ultrasound transducer of the invention; and

FIG. 3 shows a partial region of the ultrasound transducer of another embodiment.

### DESCRIPTION OF THE PREFERRED EMBODIMENTS

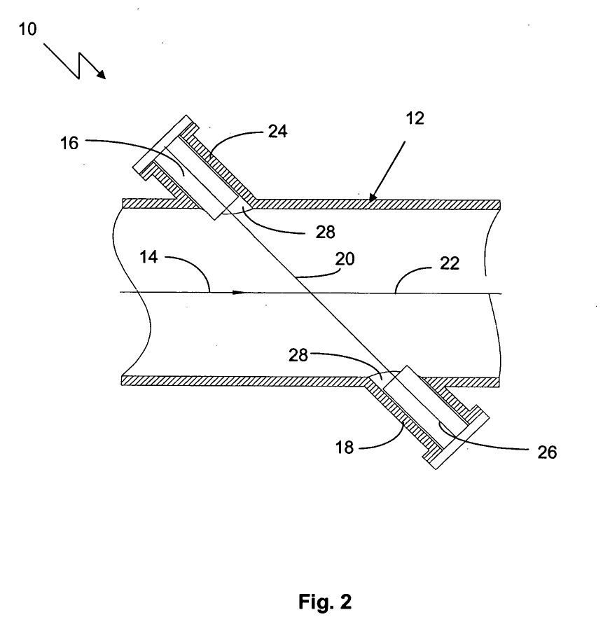

FIG. 2 shows a metering layout 10 and illustrates the measurement principle employed by the present invention, for example in an ultrasound gas flow meter. A gas flows in a pipeline 12 in a flow direction 14. Identically configured ultrasound transducers 16 and 18 are arranged in pipeline 12 and define a metering path 20. Ultrasound transducers 16, 18 suitably convert electrical signals into ultrasound, and vice versa, for sending and receiving of ultrasound. The metering path 20 is oriented at an angle other than 90° to a longitudinal axis 22 of pipeline 12 so that the ultrasonic signals sent out in opposite directions along the metering path 20 have different transit times due to gas flow 14 in the pipe. The flow velocity and, thus, the volume flow rate of the gas can be determined from the transit time difference and the geometry of the system.

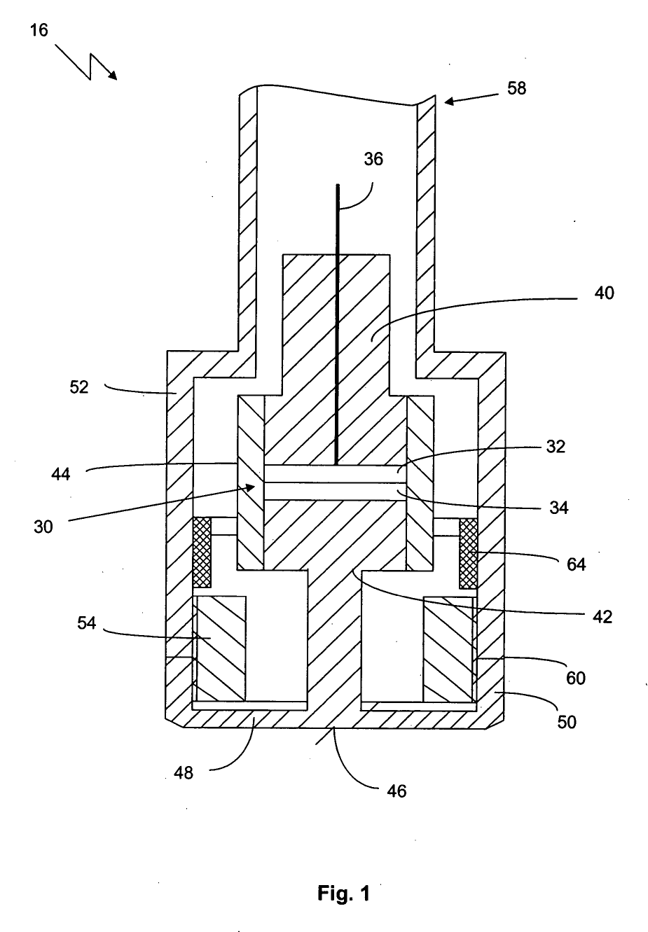

Referring to FIG. 1, an ultrasound transducer 16 has an ultrasound generating element 30, which can comprise two piezoceramics 32, 34 coupled to an insulated electrical conductor 36. The piezoelectric element 30 is clamped between two cylindrical holders 40 and 42 which are held together by a clamp 44. An end face 46 of holder 42 serves as the sending and/or receiving surface by which the ultrasonic signals are emitted or received.

To enlarge end surface 46, the end face is defined by a plate 48, hereinafter also referred to as a membrane 48. To emit ultrasonic signals, membrane 48 oscillates in response to ultrasonic oscillations generated by piezoelectric element 30 and transmitted via rigid holder 42. To receive ultrasonic signals, the signal processing is reversed. Membrane 48 picks up the ultrasonic oscillations, which are transmitted via holder 42 to the piezoelectric element 30, which converts the picked-up oscillations into corresponding electric signals.

Referring to FIGS. 1 and 2, membrane 48 has an angled outer edge portion 50 that is joined to a substantially cylindrical housing 52 which houses the earlier mentioned signal conducting and signal processing components.

At its inside, the outer edge portion 50 of membrane 48 is coupled to a mass ring 54, preferably with a threaded connection. The mass ring is coupled to membrane 48 only along its outwardly facing surface, i.e. only along the outer edge portion 50. This provides the membrane 48 with the largest possible oscillating surface. Thus, on the inside, mass ring 54 is separated from the membrane 48 by an L-shaped gap.

Mass ring 54 projects past the outer edge portion 50 into which it is threaded so that the mass ring can also be joined to a housing 52, preferably also by means of a threaded connection. Since the housing can be secured in the flow meter in suitable manner, not further shown, for example with a flange positioned at housing end 58 away from membrane 48, the signal conducting and signal processing components are also supported by mass ring 54.

To achieve an absolute seal and tightness of the transducer 16, in one embodiment of the invention, housing 52 and outer edge portion 50 are connected by a weld 60. [00261 To further reduce parasitic oscillations, a dampening element 64 is placed inside housing 52. It is made of an elastic material, such as rubber. The dampening element is configured as a ring, lies against the inside of housing 52, and is advantageously arranged close to mass ring 54.

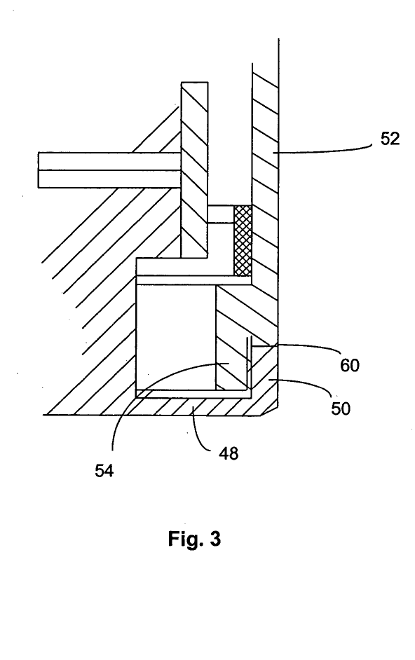

In another embodiment, shown in FIG. 3, the mass ring 54 can also be made of a single piece with the housing 52.

1. An ultrasound transducer comprising a housing with at least one piezoelectric element, a membrane for emitting and receiving ultrasound, a mass ring coupled to the membrane for dampening undesirable resonances, the mass ring having an outwardly facing surface connected to an outer edge portion of the membrane and to an inside of the housing, and a dampening element having a ring-shaped configuration, being connected to the inside of the housing and arranged proximate the mass ring.

2. An ultrasound transducer according to claim 1 wherein the transducer comprises a longitudinally vibrating oscillator.

3. An ultrasound transducer according to claim 1 wherein the mass ring is threadably connected to the membrane.

4. An ultrasound transducer according to claim 1 wherein the membrane and the housing are welded together proximate the mass ring.

5. An ultrasound transducer according to claim 1 wherein the dampening element comprises an elastic material.

6. An ultrasound transducer according to claim 5 wherein the elastic material is a rubber material.

7. An ultrasound transducer according to claim 1 wherein the mass ring is threadably attached to the housing.

8. An ultrasound transducer according to claim 1 wherein the mass ring and the housing comprise a single piece.
